(19) United States
(12) Patent Application Publication
(10) Pub. No.: US 2024/0343982 A1
(43) Pub. Date: Oct. 17, 2024

(54) Method for Improving Oil Quality and Increasing Yield of Low-carbon Olefins by Utilizing Bio-Oil Catalytic Cracking

(71) Applicant: REZEL CATALYSTS (SHANGHAI) CO., LTD., Shanghai (CN)

(72) Inventors: Runsheng ZHUO, Leshan (CN); Zongbo SHI, Leshan (CN); Xinsheng LIU, Leshan (CN); Qing ZHANG, Leshan (CN); Zesong HU, Leshan (CN); Mingyang LI, Leshan (CN)

(21) Appl. No.: 18/748,329

(22) Filed: Jun. 20, 2024

Related U.S. Application Data
(63) Continuation of application No. 17/056,426, filed on Nov. 18, 2020.

(51) Int. Cl. C10G 3/00; B01J 29/08; B01J 29/40

(52) U.S. Cl. CPC C10G 3/49; B01J 29/084; B01J 29/40; C10G 2300/1011; C10G 2300/202; C10G 2300/301; C10G 2300/305; C10G 2300/308; C10G 2300/70; C10G 2400/20

(57) ABSTRACT

The Invention discloses a method for improving the quality of oil products and increasing the yield of low-carbon olefins by catalytic cracking of bio-oil, which takes bio-oil or mixed oil of bio-oil and hydrocarbon oil as raw oil for catalytic cracking reaction. With this method, the octane number of the gasoline in product is obviously increased, simultaneously, the content of propylene and other low-carbon olefins in product is also improved.

### CROSS REFERENCE TO RELATED APPLICATIONS

This application is a continuation of U.S. patent application Ser. No. 17/056,426, filed on Nov. 18, 2020, and the entire contents of which are incorporated herein by reference.

### FIELD OF THE INVENTION

The Invention relates to the field of new energy and petroleum refining, in particular to method for improving the quality of oil products and increasing the yield of low-carbon olefins by utilizing bio-oil catalytic cracking.

### BACKGROUND OF THE INVENTION

With the petroleum exploitation of oil year by year, oil reserves are gradually declining. According to the statistics of BP Statistical Review of World Energy in 2018, petroleum can only be produced in 50.2 years at the current oil exploitation speed. Petroleum is not only an important energy substance, but also the most important basic raw material of chemical products, among which gasoline, diesel oil, kerosene and other products produced by petroleum are important energy substances, and ethylene, propylene and aromatic hydrocarbons are important chemical raw materials. Fossil energy based on petroleum is non-renewable, so it is of great practical significance to use renewable raw materials to produce chemical products such as gasoline, diesel oil, ethylene and propylene. In addition, if a country is subject to economic sanctions or military blockade, it is of great strategic significance to use bio-oil for catalytic cracking.

Bio-oil is a kind of renewable energy with a wide range of sources, which comes from photosynthesis of plants directly or indirectly. Bio-oil can be esterified with methanol or ethanol to form fatty acid methyl ester or ethyl ester, namely biodiesel. CN105586154A discloses a continuous esterification method for preparing biodiesel from waste oil and fat, wherein biodiesel is prepared through continuous esterification reaction of methanol and waste oil and fat. CN102027095A discloses a combined method for producing diesel fuel from biomaterials, and products, application and equipment related to the method, wherein paraffin is produced through Fischer-Tropsch reaction on the one hand, and bio-oil and fat undergo catalytic hydrodeoxygenation on the other hand, and those two hydrocarbon streams are combined and distilled to produce biodiesel products. Biodiesel is featured by good environmental protection performance, good engine starting performance and good fuel performance, but itis only suitable for diesel engines, and it has high oxygen content and low combustion calorific value.

Through catalytic cracking, bio-oil can also be converted into gasoline products, which is an important method for comprehensive utilization of bio-oil. CN102676201A discloses a method for preparing high-quality gasoline from cracked bio-oil, wherein crude bio-oil, lignocellulose, lignin, lignin-derived phenolic monomer or/and dimer thereof, cellulose and cellulose-derived furan compound are used as raw materials, and under the catalysis of Ni/HMFI catalyst, they undergo hydrodeoxygenation conversion by one step to produce hydrocarbon fuel. CN102676202A discloses a method for preparing high-quality gasoline and diesel oil from lignin pyrolysis oil, wherein lignin pyrolysis oil, crude bio-oil, lignin and lignin-derived phenolic monomer or/and dimers are used as raw materials, and the raw materials are converted into C6-C9 gasoline and C12-C20 diesel hydrocarbon fuels with adjustable proportions by one step under the catalytic action of Ni-based or Pd-based catalysts supported on zeolite. CN1916135 discloses a method for producing fuel oil from bio-grease, wherein bio-grease is used to produce liquefied gas, gasoline and diesel oil products under the catalysis of solid acid catalyst. The total weight percentage of liquefied gas, gasoline and diesel oil can reach 88-92%, and the weight percentage of propylene content in liquefied gas can reach over 40%. CN101720349A discloses a method for preparing bio-gasoline components, wherein bio-oil is converted into gasoline components through catalytic cracking and alkylation (or catalytic polymerization) processes. CN101314724 discloses a combined catalytic conversion method of bio-oil and mineral oil, wherein the bio-oil and mineral oil are contacted with a catalyst containing modified Beta zeolite in a composite reactor for catalytic cracking reaction, and the target products such as low-carbon olefins, gasoline, diesel oil and heavy oil are separated by fractionation.

The conversion of bio-oil to low-carbon olefin through catalytic cracking process is an indispensable process for preparation of chemical basic raw materials from bio-oil. CN107964419A discloses a process of bio-grease, which comprises the following steps: contacting bio-grease with a catalytic cracking catalyst in reactor and performing catalytic cracking reaction to obtain a catalytic cracking product. The process can produce more low-carbon olefins and improve the utilization rate of hydrocarbons. CN102452887A discloses a method for preparing low-carbon olefins from bio-grease, which includes hydrogenation process and catalytic cracking process, and the method can obviously improve the yield of low-carbon olefins. CN101747134A discloses a method for producing low-carbon olefins by biomass catalytic cracking of biomass. The method provides a biomass utilization method on the one hand, a catalytic cracking catalyst for biomass feedstock to produce low-carbon olefins and a catalyst preparation method thereof on the other hand. CN101314718B discloses a method for improving the yield of low-carbon olefins during catalytic conversion reaction of bio-grease, wherein bio-grease is added to a catalytic conversion reactor and then converted to ethylene, propylene and butene by reaction over a catalyst containing MFI zeolite. CN102712850B discloses a method for preparing hydrocarbon products from bio-oil and/or kerosene, wherein coal and/or biomass is used as raw materials to produce short-chain hydrocarbons, with low conversion rate and high coke content in the products. CN109575978A discloses a processing method of bio-grease, wherein raw materials containing bio-grease fed into a catalytic cracking reactor to contact with a catalytic cracking catalyst for catalytic cracking reaction, wherein the catalytic cracking catalyst contains zeolite and metal oxide with adsorption function. The processing method can improve product distribution, reduce coke yield, and improve the yield of low-carbon olefins and light aromatic hydrocarbon. CN107460005A discloses a method and a device for producing aromatic hydrocarbons and olefins by catalytic hydrogenation coupled catalytic cracking of bio-oil, wherein biomass is thermally cracked to bio-oil, and the bio-oil undergoes hydrogenation and catalytic cracking to produce aromatic hydrocarbons and olefins.

In addition to producing biodiesel, gasoline and low-carbon olefins, bio-oil can also be used to produce alkanes, hydrogen and other products. CN101558135 discloses a fluidized catalytic cracking method of oxygenated compounds, wherein the contact time between oxygenated hydrocarbon compounds and fluidized cracking catalytic materials is less than 3 seconds, and the cracking products in the process are mainly CO₂, CO, H₂, aromatic hydrocarbons and coke. CN104722329A discloses a catalyst for preparing alkane by catalytic hydrogenation of bio-grease. With 10%˜50% of base metal nickel (Ni) salt, molybdenum (Mo) salt, cobalt (Co) salt and tungsten (W) salt as active components, and modified zeolite/alumina as catalyst carrier, the non-sulfurized bio-grease hydrofining catalyst reduces the production cost and is conducive to alleviating the crisis of petrochemical energy shortage. CN108554418A discloses a Ni—B—La catalyst for hydrogen production by catalytic reforming of bio-oil and a preparation method. The catalyst is featured by wide raw material sources, low price, good sintering and carbon deposition resistance, strong stability, high reaction activity, long service life, high conversion rate of bio-oil and high hydrogen yield. CN106064089A discloses a renewable catalyst for hydrogen production by catalytic reforming of bio-oil and a preparation method thereof. The catalyst is renewable and stable in hydrogen production process, and can be regenerated and recycled for many times.

### Technical Problem

The Invention aims to provide a method for improving the quality of oil products and improving the yield of low-carbon olefins by using bio-oil catalytic cracking. Bio-oil and/or mixed oil of bio-oil and hydrocarbon oil are used as catalytic cracking raw materials. Through catalytic cracking process under the action of a catalyst, product quality is improved.

### Technical Solution

In order to solve the above technical problems, one set of technical solutions provided by the Invention is a method for improving oil quality and increasing the yield of low-carbon olefins by catalytic cracking of bio-oil, wherein bio-oil or mixed oil of bio-oil and hydrocarbon oil is used as raw oil for catalytic cracking reaction.

Said bio-oil has a hydrogen/carbon molar ratio of 1.75-1.95 and a carbon/oxygen molar ratio of 8-9.5.

Said biological oil includes palm oil, peanut oil, soybean oil and/or sewer oil.

Said hydrocarbon oil includes straight-run distillate oil, atmospheric residue and/or vacuum residue, and preferably, said hydrocarbon oil includes coker gas oil, deasphalted oil, foot oil from raw paraffin and/or extract oil.

Said catalytic cracking reaction comprises three parts: a reaction-regeneration system, a fractionation system and an absorption-stabilization system.

Said catalytic crack reaction particularly comprises of the following steps: introducing biological oil or mixed oil of biological oil and hydrocarbon oil as raw oil into a catalytic crack or cracking device for catalytic cracking or cracking reaction, obtaining cracking products under the action of a catalyst, carrying out cyclone separation of the catalyst from the cracking products, and separating the cracking products by the fractionation system followed by the absorption-stabilization system. Preferably, the mass ratio of catalyst to the raw oil is 4-12; preferably, the outlet temperature of catalytic reaction is 490˜580° C.

Said catalyst consists of zeolite, inorganic matrix, clay and binder, wherein the content of zeolite is 25%-40%; preferably, the zeolite consists of Y-type zeolite and ZSM-5 zeolite.

Said Y-type zeolite is a USY zeolite, or a Y-type zeolite and a USY-type zeolite modified by mixing one or more elements of rare earth, phosphorus and alkaline earth metals. Furthermore, said ZSM-5 zeolite content is no less than 3% of the total zeolite; preferably, the mole ratio of SiO₂/Al₂O₃ of the ZSM-5 zeolite is 20-50; Furthermore, the ZSM-5 zeolite is a ZSM-5 zeolite modified with phosphorus and/or rare earth elements.

With the above catalyst and method, high octane gasoline, diesel oil, kerosene, low-carbon olefin and other products can be obtained.

The Invention also provides another technical solution, namely, a method for improving the yield of ethylene and propylene by using bio-oil catalytic cracking/thermal cracking, which comprises of the following steps: using bio-oil or mixed oil of bio-oil and hydrocarbon as raw materials for catalytic cracking/thermal cracking, and producing ethylene, propylene, gasoline and diesel oil through catalytic cracking/thermal cracking reaction under the action of catalyst, wherein the total yield of ethylene and propylene is more than 30%.

The bio-oil has a hydrogen/carbon molar ratio of 1.75-3:1 and a carbon/oxygen molar ratio of 8-12:1, and the bio-oil includes palm oil, peanut oil, soybean oil and illegal cooking oil.

Calculated on a dry basis, the catalyst comprises of 40%-60% of modified 10 MR zeolite, 20%-40% of clay, 10%-20% of alumina matrix and 1%-12% of binder.

The modified 10 MR zeolite is a 10 MR zeolite modified by IIIA group and phosphorus element through a post-modification method, and has 10-100:1 molar ratio of SiO₂/Al₂O₃, 1-5% content of P₂O₅, and 0.1-3% content of oxides of IIIA group elements.

The Invention discovers that 1.75-3:1 molar ratio of hydrogen to carbon and 8-12:1 molar ratio of carbon to oxygen of bio-oil will lead to high yield of ethylene and propylene in the cracked product through catalytic cracking/thermal cracking. In addition, the Invention optimizes the design of catalyst and catalytic process, and creatively discovers that the total yield of ethylene and propylene can exceed 30% under the conditions that alumina is selected for cracking reaction, IIIA group and phosphorus modified 10 MR zeolite is used for cracking reaction, the content of modified 10 MR zeolite is 40%˜60%, alumina matrix contains 10%˜20% catalyst, C4 hydrocarbon and light naphtha are recycled. The Invention is mainly used in the field of new energy, and aims to provide a method for improving the yield of ethylene and propylene by catalytic cracking/thermal cracking of bio-oil or mixed oil of bio-oil and hydrocarbons on the basis of the prior art, wherein the mixed oil of bio-oil is used as a raw material for catalytic cracking/thermal cracking, and catalytic reaction is carried out by the traditional catalytic cracking/thermal cracking process under the action of a catalyst to obtain products such as ethylene and propylene.

It particularly includes the following steps: bio-oil or mixed oil of bio-oil and hydrocarbon is injected into a catalytic cracking/thermal cracking device for catalytic cracking/thermal cracking reaction and cracked under the action of a catalyst to produce cracking products like gasoline, diesel oil, liquefied gas, dry gas and slurry. After cyclone separation of cracked products and catalysts, the separated catalyst is regenerated in a regenerator and then the products are separated into gasoline, diesel oil, kerosene, butane, butene, light naphtha, ethylene and propylene by fractionation system and absorption-stabilization system, and some separated butane, butene and light naphtha are mixed with the feed and then recycled.

The outlet temperature of catalytic cracking/thermal cracking reaction is 550-650° C., the mass ratio of catalyst to raw material is 7.5-20:1, and the weight hourly space velocity (WHSV) based on raw material is 0.2-20 h⁻¹.

The content of bio-oil in the mixed oil of bio-oil and hydrocarbons exceeds 85%, and hydrocarbons include one or more of straight distillate oil, atmospheric residue, vacuum residue, coker gas oil, deasphalted oil, foot oil from raw paraffin, extract oil, butane, butene, naphtha, plastic, resin and polyolefin.

Said inorganic matrix is alumina and/or modified alumina.

Said binder is an alumina binder and/or a silica binder.

Catalytic cracking/thermal cracking herein refers to catalytic cracking or catalytic thermal cracking.

### Beneficial Effects

With this Invention, the octane number of the gasoline in the product is obviously increased, and the content of propylene and other low-carbon olefins in the product is also raised.

### MOST PREFERRED EMBODIMENTS OF THE INVENTION

### Embodiment 1

Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension, and then add 2 kg (dry-basis) pseudo-boehmite. Adjust pH of the suspension to 2.5˜3.5 with HCl, so that the pseudo-boehmite can experience a gelation reaction. After stirring for 30 min, add azeolite suspension containing 3.55 kg (dry basis) RE/USY (RE₂O₃=4%) and 0.25 kg (dry basis) P/ZSM-5 (molar ratio of SiO₂/Al₂O₃=27, P₂O₅=3%). Keep blending for 30 min until the solid content of the suspension slurry obtained is 35%; Homogenize the suspension before spray-drying, and then calcine the spray-dried material at 500° C. for 2 hours. The bio-oil fluidized catalytic cracking catalyst Bio-FCC-1 is obtained, which has a wear index of 0. 7wt %/h and a specific surface area of 309 m²/g.

### EMBODIMENTS OF THE INVENTION

### Detailed Description of the Preferred Embodiments

In the following, the claims of the Invention will be further described in detail with reference to specific embodiments.

In the following embodiments and comparative examples, the specific surface area of samples is measured by BET low temperature nitrogen adsorption method, the elemental composition of samples is measured by X-ray fluorescence spectrometer, and the wear index of samples is measured by wear index analyzer. For other analysis, refer to the National Standard of Method for Test of Petroleum and Petroleum Products (Standards Press of China, 1989).

### Comparative Example 1

Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension and then add 2 kg (dry-basis) pseudo-boehmite. Adjust pH of the suspension to 2.5˜3.5 with HCl, so that the pseudo-boehmite can experience a gelation reaction. After stirring for 30 min, add a zeolite suspension containing 3.5 kg (dry basis) RE/USY (RE₂O₃=4%). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst FCC-1 is obtained, which has a wear index of 0.9 wt %/h and a specific surface area of 296 m²/g.

### Comparative Example 2

Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension, and then add 2 kg (dry-basis) pseudo-boehmite. Adjust pH of the suspension to 2.5˜3.5 with HCl, so that the pseudo-boehmite can experience a gelation reaction. After stirring for 30 min, add a zeolite suspension containing 3.5 kg (dry basis) P/ZSM-5 (molar ratio of SiO₂/Al₂O₃=27, P₂O₅=3%). Keep blending for 30 min until the solid content of the suspension slurry obtained is 35%; Homogenize the suspension slurry before spray-drying, and then calcine the spray-dried material at 500° C. for 2 hours. The catalytic cracking catalyst FCC-2 is obtained, which has a wear index of 2.4 wt %/h and a specific surface area of 176 m²/g.

### Embodiment 2

Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension, and then add 2 kg (dry-basis) pseudo-boehmite. Adjust pH of the suspension to 2.5˜3.5 with HCl, so that the pseudo-boehmite can experience a gelation reaction. After stirring for 30 min, add a zeolite suspension containing 3.55 kg (dry basis) RE/Mg/P/USY (RE₂O₃=4%, MgO=0.3%, P₂O₅=0.4%) and 0.25 kg (dry basis) RE/P/ZSM-5 (molar ratio of SiO₂/Al₂O₃, P₂O₅=3%, RE2O3=0.3%). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst Bio-FCC-2 is obtained, which has a wear index of 0.9 wt %/h and a specific surface area of 284 m²/g.

### Embodiment 3

Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension, and then add 2 kg (dry-basis) pseudo-boehmite. Adjust pH of the suspension to 2.5˜3.5 with HCl, so that the pseudo-boehmite can experience a gelation reaction. After stirring for 30 min, add a zeolite suspension containing 3.55 kg (dry basis) USY and 0.25 kg (dry basis) P/ZSM-5 (molar ratio of SiO₂/Al₂O₃=50, P₂O₅=3%). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst Bio-FCC-3 is obtained, which has a wear index of 0.9 wt %/h and a specific surface area of 272 m²/g.

### Embodiment 4

Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension, and then add 2 kg (dry-basis) pseudo-boehmite. Adjust pH of the suspension to 2.5˜3.5 with HCl, so that the pseudo-boehmite can experience a gelation reaction. After stirring for 30 min, add a zeolite suspension containing 1.5 kg (dry basis) silica sol and 0.9 kg (dry basis) RE/USY (RE₂O₃=3. 5%) and 1.2 kg (dry basis) P/ZSM-5 (molar ratio of SiO₂/Al₂O₃=50, P₂O₅=3%). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst Bio-FCC-4 is obtained, which has a wear index of 1.2wt %/h and a specific surface area of 264 m²/g.

In described embodiments and comparative examples, the catalytic cracking reaction is assessed with miniature fluidized bed reactor (ACE) and supporting gas chromatography, while research octane number (RON) is analyzed with Agilent gas chromatography 7980A. See Table 1 for physical and chemical properties of vacuum distillate oil, and see Table 2 for C/O and H/C molar ratios of palm oil, peanut oil, soybean oil, sewer oil and furfural.

TABLE 1
Physical and Chemical Properties of Vacuum Distillate Oil

| Item | Result |
|---|---|
| Density, 15 degC., kg/m³ | 901 |
| Sulfur content, ppmw | 2270 |
| Nitrogen content, ppmw | 845 |

Distillation range (Deg C.) ASTM D-1160

| | |
|---|---|
| 15% | 229° C. |
| 10% | 335° C. |
| 30% | 392° C. |
| 50% | 425° C. |
| 70% | 451° C. |
| 90% | 499° C. |
| 95% | 535° C. |
| H element content (wt %) | 13.1 |
| Ni, ppmw | 1.8 |
| V, ppmw | 0.27 |
| Fe, ppmw | 1.5 |
| Na, ppmw | <10 |
| Residual carbon (wt %) | 3.59 |

TABLE 2
Properties of Bio-oil

| | Molar ratio of | Molar ratio of |
|---|---|---|
| Bio-oil | carbon/oxygen | oxygen/carbon |
| Palm oil | 8.24 | 1.80 |
| Peanut oil | 9.21 | 1.90 |
| Soybean oil | 8.93 | 1.81 |
| Sewer oil | 9.06 | 1.82 |
| Furfural | 2.50 | 0.80 |

### Comparative Experimental Example 1

Catalyst and catalytic cracking raw oil are FCC-1 catalyst and vacuum gas oil respectively.

Process conditions: evaluated on ACE, reaction temperature is 510° C., catalyst/oil ratio is 5.6, catalyst loading is 9 g, oil feeding speed is 1.2 g/min, the pretreatment temperature of the catalyst is 814° C., and the catalyst is treated with 100% steam for 10 hours.

### Comparative Experimental Example 2

Catalyst and catalytic cracking raw oil are FCC-2 catalyst and vacuum gas oil respectively.

Process conditions: evaluated on ACE, reaction temperature is 510° C., catalyst-oil ratio is 5.6, catalyst loading is 9 g, oil feeding speed is 1.2 g/min, the pretreatment temperature of the catalyst is 814° C., and the catalyst is treated with 100% steam for 10 hours.

### Comparative Experimental Example 3

Catalyst and catalytic cracking raw oil are FCC-1 catalyst, 80% vacuum gas oil with 20% furfural respectively.

Process conditions: evaluated on ACE, reaction temperature is 510° C., catalyst/oil ratio is 5.6, catalyst loading is 9 g, oil feeding speed is 1.2 g/min, the pretreatment temperature of the catalyst is 814° C., and the catalyst is treated with 100% steam for 10 hours.

### Experimental Example 1

Catalyst and catalytic cracking raw oil are Bio-FCC-1 catalyst and vacuum gas oil respectively.

Process conditions: evaluated on ACE, reaction temperature is 510° C., catalyst/oil ratio is 5.6, catalyst loading is 9 g, oil feeding speed is 1.2 g/min, the pretreatment temperature of the catalyst is 814° C., and the catalyst is treated with 100% steam for 10 hours.

### Experimental Example 2

Catalyst and catalytic cracking raw oil are Bio-FCC-1 catalyst and palm oil respectively.

Process conditions: evaluated on ACE, reaction temperature is 510° C., catalyst/oil ratio is 5.6, catalyst loading is 9 g, oil feeding speed is 1.2 g/min, the pretreatment temperature of the catalyst is 814° C., and the catalyst is treated with 100% steam for 10 hours.

### Experimental Example 3

Catalyst and catalytic cracking raw oil are Bio-FCC-1 catalyst, 50% palm oil with 50% vacuum gas oil respectively.

Process conditions: evaluated on ACE, reaction temperature is 510° C., catalyst/oil ratio is 5.6, catalyst loading is 9 g, oil feeding speed is 1.2 g/min, the pretreatment temperature of the catalyst is 814° C., and the catalyst is treated with 100% steam for 10 hours.

### Experimental Example 4

Catalyst and catalytic cracking raw oil are Bio-FCC-2 catalyst and peanut oil respectively.

Process conditions: evaluated on ACE, reaction temperature is 510° C., catalyst/oil ratio is 5.6, catalyst loading is 9 g, oil feeding speed is 1.2 g/min, the pretreatment temperature of the catalyst is 814° C., and the catalyst is treated with 100% steam for 10 hours.

### Experimental Example 5

Catalyst and catalytic cracking raw oil are Bio-FCC-3 catalyst and soybean oil respectively.

Process conditions: evaluated on ACE, reaction temperature is 490° C., catalyst/oil ratio is 4, catalyst loading is 9 g, oil feeding speed is 1.2 g/min, the pretreatment temperature of the catalyst is 814° C., and the catalyst is treated with 100% steam for 10 hours.

### Experimental Example 6

Catalyst and catalytic cracking raw oil are Bio-FCC-4 catalyst and sewer oil respectively.

Process conditions: evaluated on ACE, reaction temperature is 580° C., catalyst/oil ratio is 12, catalyst loading is 9 g, oil feeding speed is 1.2 g/min, the pretreatment temperature of the catalyst is 814° C., and the catalyst is treated with 100% steam for 10 hours.

ACE evaluation results of the above experimental examples are shown in Table 3:

TABLE 3
Performance of Catalytic Cracking of Samples in Embodiments and Comparative Examples

| | Comparative | Comparative | Comparative | | | | | | |
|---|---|---|---|---|---|---|---|---|---|
| | experimental | experimental | experimental | Experimental | Experimental | Experimental | Experimental | Experimental | Experimental |
| | example 1 | example 2 | example 3 | example 1 | example 2 | example 3 | example 4 | example 5 | example 6 |
| Conversion | 85.30 | 54.77 | 86.43 | 85.22 | 88.83 | 87.29 | 88.34 | 85.38 | 85.92 |
| rate, wt % | | | | | | | | | |
| Coke, wt % | 9.10 | 4.72 | 10.76 | 9.00 | 4.41 | 6.70 | 4.45 | 3.30 | 7.47 |
| Dry gas, | 2.12 | 5.80 | 2.24 | 2.05 | 1.86 | 1.92 | 1.98 | 1.80 | 3.93 |
| wt % | | | | | | | | | |
| Ethylene, | 0.65 | 3.11 | 0.69 | 0.83 | 1.05 | 0.96 | 1.03 | 1.01 | 2.67 |
| wt % | | | | | | | | | |
| Propylene, | 5.15 | 10.81 | 5.50 | 6.53 | 8.35 | 7.57 | 8.20 | 7.99 | 13.82 |
| wt % | | | | | | | | | |
| Butene, | 4.70 | 8.35 | 5.03 | 5.88 | 7.53 | 6.97 | 7.55 | 7.31 | 15.01 |
| wt % | | | | | | | | | |
| Gasoline, | 54.53 | 23.59 | 44.48 | 50.19 | 46.65 | 48.19 | 47.21 | 46.61 | 32.23 |
| wt % | | | | | | | | | |
| Diesel oil, | 10.56 | 21.23 | 9.50 | 10.39 | 8.87 | 9.46 | 9.14 | 11.53 | 10.92 |
| wt % | | | | | | | | | |
| Slurry, | 4.14 | 24.00 | 4.08 | 4.38 | 2.30 | 3.25 | 2.52 | 3.09 | 3.16 |
| wt % | | | | | | | | | |
| Liquefied | 19.54 | 20.66 | 19.30 | 23.98 | 25.38 | 24.72 | 25.28 | 24.35 | 32.58 |
| gas, wt % | | | | | | | | | |
| H₂O + CO + | 0.00 | 0.00 | 9.63 | 0.00 | 10.53 | 5.77 | 9.41 | 9.33 | 9.70 |
| CO₂ | | | | | | | | | |
| Octane | 89.8 | — | 90.3 | 91.1 | 92.7 | 92.2 | 92.9 | 93.1 | 99.4 |
| number of | | | | | | | | | |
| gasoline | | | | | | | | | |

### Embodiment 5

Preparation of catalyst: Add 2.1 kg (dry-basis) kaolinite and 0.4 kg (dry-basis) alumina sol to 3.5 kg deionized water while stirring, and stir at high speed for 1 h. Wait for the kaolinite to be completely distributed in the suspension, and then add 3.5 kg (dry-basis) industrial porous pseudo-boehmite. Adjust pH of the suspension to 2.5˜3.5 with HCl, so that the pseudo-boehmite can experience a gelation reaction. After stirring for 30 minutes, add a 4 kg zeolite suspension containing Al/P/ZSM-5 (Al₂O₃=0.6%, P₂O₅=3%, SiO₂/Al₂O₃=27 for modification). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst Bio-DCC-1 is obtained.

The wear index of the catalyst Bio-DCC-1 in Embodiment 5 is 0. 7wt %/h and the specific surface area is 209 m²/g.

Catalytic cracking/thermal cracking raw oil: palm oil.

Process conditions: evaluated on ACE, reaction temperature is 600° C., catalyst/oil ratio is 10, catalyst loading is 9 g, oil feeding speed is 1.2 g/min, and 15% C4 hydrocarbon and light naphtha are recycled. The pretreatment temperature of the catalyst is 814° C., and the catalyst is treated with 100% steam for 10 hours. ACE evaluation results are shown in Table 4.

### Embodiment 6

Preparation of catalyst: Add 1.9 kg (dry-basis) kaolinite and 0.1 kg (dry-basis) alumina sol to 3.5 kg deionized water while stirring, and stir at high speed for 1 h. Wait for the kaolinite to be completely dispersed in the suspension, and then add 2.5 kg (dry-basis) industrial porous pseudo-boehmite. Adjust pH of the suspension to 2.5˜3.5 with HCl, so that the pseudo-boehmite can experience a gelation reaction. After stirring for 30 minutes, add a 5.5 kg zeolite suspension containing B/P/ZSM-5 (B₂O₃=0.6%, P₂O₅=3%, SiO₂/Al₂O₃=39 for modification). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst Bio-DCC-2 is obtained.

The wear index of the catalyst Bio-DCC-2 in Embodiment 6 is 2.6 wt %/h and the specific surface area is 214 m²/g.

Catalytic cracking/thermal cracking raw oil: palm oil.

Process conditions: evaluated on ACE, reaction temperature is 600° C., catalyst/oil ratio is 10, catalyst loading is 9 g, oil feeding speed is 1.2 g/min, and 15% C4 hydrocarbon and light naphtha are recycled. The pretreatment temperature of the catalyst is 814° C., and the catalyst is treated with 100% steam for 10 hours. ACE evaluation results are shown in Table 4.

### Embodiment 7

Preparation of catalyst: Add 2.6 kg (dry-basis) kaolinite and 0.4 kg (dry-basis) alumina sol to 3.5 kg deionized water while stirring, and stir at high speed for 1 h. Wait for the kaolinite to be completely dispersed in the suspension, and then add 3 kg (dry-basis) industrial porous pseudo-boehmite. Adjust pH of the suspension to 2.5˜3.5 with HCl, so that the pseudo-boehmite can experience a gelation reaction. After stirring for 30 minutes, add a 4 kg zeolite suspension containing Ga/P/ZSM-5 (Ga₂O₃=0.6%, P₂O₅=3%, SiO₂/Al₂O₃=39 for modification). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst Bio-DCC-3 is obtained.

The wear index of the catalyst Bio-DCC-3 in Embodiment 7 is 0.7 wt %/h and the specific surface area is 209 m²/g.

Catalytic cracking/thermal cracking raw oil: 90% palm oil with 10% vacuum gas oil.

Process conditions: evaluated on ACE, reaction temperature is 600° C., catalyst/oil ratio is 10, catalyst loading is 9 g, oil feeding speed is 1.2 g/min, and 15% C4 hydrocarbon and light naphtha are recycled. The pretreatment temperature of the catalyst is 814° C., and the catalyst is treated with 100% steam for 10 hours. ACE evaluation results are shown in Table 4.

### Embodiment 8

Preparation of catalyst: Add 2.6 kg (dry-basis) kaolinite and 0.4 kg (dry-basis) alumina sol to 3.5 kg deionized water while stirring, and stir at high speed for 1 h. Wait for the kaolinite to be completely dispersed in the suspension, and then add 3 kg (dry-basis) industrial porous pseudo-boehmite. Adjust pH of the suspension to 2.5˜3.5 with HCl, so that the pseudo-boehmite can experience a gelation reaction. After stirring for 30 minutes, add a 4 kg zeolite suspension containing Ga/P/ZSM-11 (Ga₂O₃=0.6%, P₂O₃=3%, SiO₂/Al₂O₃3=61 for modification). Keep blending for 30 min until the solid content of the suspension slurry obtained is 35%; Homogenize the size before spray-drying, and then calcine the spray-dried material at 500° C. for 2 hours. The bio-oil fluidized catalytic cracking catalyst Bio-DCC-4 is obtained.

Catalytic cracking/thermal cracking raw oil: palm oil.

Process conditions: evaluated on ACE, reaction temperature is 560° C., catalyst/oil ratio is 7.5, catalyst loading is 9 g, oil feeding speed is 1.2 g/min, 10% C4 hydrocarbon and light naphtha are recycled, the pretreatment temperature of the catalyst is 814° C., and the catalyst is treated with 100% steam for 10 hours. ACE evaluation results are shown in Table 4.

### Embodiment 9

Catalyst Bio-DCC-3 in Embodiment 3 is selected as the catalyst.

Catalytic cracking/thermal cracking raw oil: peanut oil.

Process conditions: evaluated on ACE, reaction temperature is 560° C., catalyst/oil ratio is 7.5, catalyst loading is 9 g, oil feeding speed is 1.2 g/min, 10% C4 hydrocarbon and light naphtha are recycled. The pretreatment temperature of the catalyst is 814° C., and the catalyst is treated with 100% steam for 10 hours. ACE evaluation results are shown in Table 4.

### Embodiment 10

Catalyst Bio-DCC-3 in Embodiment 7 is selected as the catalyst.

Catalytic cracking/thermal cracking raw oil: soybean oil.

Process conditions: evaluated on ACE, reaction temperature is 560° C., catalyst/oil ratio is 7.5, catalyst loading is 9 g, oil feeding speed is 1.2 g/min, 15% C4 hydrocarbon and light naphtha are recycled. The pretreatment temperature of the catalyst is 814° C., and the catalyst is treated with 100% steam for 10 hours. ACE evaluation results are shown in Table 4.

### Embodiment 11

Catalyst Bio-DCC-3 in Embodiment 7 is selected as the catalyst.

Catalytic cracking/thermal cracking raw oil: sewer oil.

Process conditions: evaluated on ACE, reaction temperature is 600° C., catalyst/oil ratio is 10, catalyst loading is 9 g, oil feeding speed is 1.2 g/min, and 15% C4 hydrocarbon and light naphtha are recycled. The pretreatment temperature of the catalyst is 814° C., and the catalyst is treated with 100% steam for 10 hours. ACE evaluation results are shown in Table 4.

### Comparative Example 3

Catalyst FCC-1 is selected as catalyst. Catalytic cracking/thermal cracking raw oil: palm oil.

Process conditions: evaluated on ACE, reaction temperature is 510° C., catalyst/oil ratio is 5.6, catalyst loading is 9 g, oil feeding speed is 1.2 g/min. The pretreatment temperature of the catalyst is 814° C., and the catalyst is treated with 100% steam for 10 hours. ACE evaluation results are shown in Table 4.

### Comparative Example 4

Catalyst FCC-1 is selected as catalyst. Catalytic cracking/thermal cracking raw oil: palm oil.

Process conditions: evaluated on ACE, reaction temperature is 560° C., catalyst/oil ratio is 7.5, catalyst loading is 9 g, oil feeding speed is 1.2 g/min. The pretreatment temperature of the catalyst is 814° C., and the catalyst is treated with 100% steam for 10 hours. ACE evaluation results are shown in Table 4.

### Comparative Example 5

Catalyst FCC-1 is selected as catalyst.

Catalytic cracking/thermal cracking raw oil: furfural.

Process conditions: evaluated on ACE, reaction temperature is 560° C., catalyst/oil ratio is 7.5, catalyst loading is 9 g, oil feeding speed is 1.2 g/min. The pretreatment temperature of the catalyst is 814° C., and the catalyst is treated with 100% steam for 10 hours. ACE evaluation results are shown in Table 4.

### Comparative Example 6

Preparation of catalyst: Add 3.1 kg (dry-basis) kaolinite and 1 kg (dry-basis) alumina sol to 3.5 kg deionized water while stirring, and stir at high speed for 1 h. Wait for the kaolinite to be completely dispersed in the suspension, and then add 2 kg (dry-basis) industrial porous pseudo-boehmite. Adjust pH of the suspension to 2.5˜3.5 with HCl, so that the pseudo-boehmite can experience a gelation reaction. After stirring for 30 min, add a zeolite suspension containing 3.5 kg (dry basis) HZSM-5 (SiO₂/Al₂O₃=27). Keep blending for 30 min until the solid content of the suspension slurry obtained is 35%; Homogenize the suspension slurry before spray-drying, and then calcine the spray-dried material at 500° C. for 2 hours. The bio-oil fluidized catalytic cracking catalyst FCC-3 is obtained.

The wear index of comparative catalyst FCC-3 is 1.0 wt %/h, and the specific surface area is 192 m²/g.

Catalytic cracking/thermal cracking raw oil: palm oil.

Process conditions: evaluated on ACE, reaction temperature is 560° C., catalyst/oil ratio is 7.5, catalyst loading is 9 g, oil feeding speed is 1.2 g/min. The pretreatment temperature of the catalyst is 814° C., and the catalyst is treated with 100% steam for 10 hours. ACE evaluation results are shown in Table 4.

TABLE 4
Catalytic Cracking/Thermal Cracking Performance of Embodiments and Comparative Examples

| | | | | | | | | Ethylene + |
|---|---|---|---|---|---|---|---|---|
| | | | | Reaction | Catalyst- | Ethylene, | Propylene, | Propylene, |
| No. | Raw oil | Cycle oil | Catalyst | temperature | oil ratio | wt % | wt % | wt % |
| Comparative | Palm oil | — | FCC-1 | 510° C. | 5.6 | 1.35 | 6.33 | 7.68 |
| example 3 | | | | | | | | |
| Comparative | Palm oil | — | FCC-1 | 560° C. | 7.5 | 1.57 | 9.83 | 11.4 |
| example 4 | | | | | | | | |
| Comparative | Furfural | | FCC-1 | 560° C. | 7.5 | 0.21 | 0.42 | 0.63 |
| example 5 | | | | | | | | |
| Comparative | Palm oil | — | FCC-3 | 560° C. | 7.5 | 3.02 | 12.33 | 15.35 |
| example 6 | | | | | | | | |
| Embodiment 5 | Peanut oil | 15% | Bio-FCC-1 | 600° C. | 10 | 8.82 | 22.8 | 31.62 |
| Embodiment 6 | Palm oil | 15% | Bio-FCC-2 | 600° C. | 10 | 8.71 | 22.91 | 31.62 |
| Embodiment 7 | 90% palm | 15% | Bio-FCC-3 | 600° C. | 10 | 8.63 | 22.57 | 31.2 |
| | oil + 10% | | | | | | | |
| | vacuum gas | | | | | | | |
| | oil | | | | | | | |
| Embodiment 8 | Palm oil | 30% | Bio-FCC-4 | 600° C. | 7.5 | 7.68 | 22.41 | 30.09 |
| Embodiment 9 | Palm oil | 30% | Bio-FCC-3 | 560° C. | 7.5 | 6.78 | 23.36 | 30.14 |
| Embodiment 10 | Soybean oil | 30% | Bio-FCC-3 | 560° C. | 7.5 | 6.92 | 23.92 | 30.84 |
| Embodiment 11 | Illegal | 15% | Bio-FCC-3 | 600° C. | 10 | 8.87 | 23.11 | 31.98 |
| | cooking oil | | | | | | | |

The embodiments above are the preferred embodiments for the Invention and not used to restrict the Invention. For the technicians of the field, various modifications and changes can be made within the ideas and principles of the Invention, and such equivalent changes or replacements are included in the range of protection in the Invention.

### INDUSTRIAL APPLICABILITY

With methods herein, the octane number of the gasoline in the product is obviously improved, and the content of propylene and other low-carbon olefins in the product is also improved, which makes good industrial sense.

1. A method for catalytic cracking of bio-oil, wherein the bio-oil or mixed oil of bio-oil and hydrocarbon oil is used as raw oil for catalytic cracking reaction; the bio-oil has hydrogen/carbon molar ratio 1.75-3:1 and carbon/oxygen molar ratio 8-12:1; wherein in the catalytic cracking reaction, a catalyst consists of zeolite, inorganic matrix, clay and binder; the zeolite consists of Y-type zeolite and ZSM-5 zeolite; wherein the ZSM-5 zeolite accounts by no less than 3% of the total zeolite; a mole ratio of SiO2/Al2O3 of ZSM-5 zeolite is 20-50:1.

2. The method according to claim 1, wherein the bio-oil has hydrogen/carbon molar ratio 1.75-1.95:1 and carbon/oxygen molar ratio 8-9.5:1.

3. The method according to claim 1, wherein the biological oil is selected from a group consisting of palm oil, peanut oil, soybean oil and sewer oil; the hydrocarbon oil is selected from a group consisting of straight distillate oil, atmospheric residual oil and vacuum residual oil.

4. The method according to claim 1, wherein the hydrocarbon oil is selected from a group consisting of coker gas oil, deasphalted oil, foot oil from raw paraffin and extract oil.

5. The method according to claim 1, wherein the catalytic cracking is comprised of: reaction-regeneration system and fractionation system.

6. The method according to claim 1, wherein the catalytic cracking reaction is as follows: biological oil or mixed oil of biological oil and hydrocarbon oil is used as raw oil and undergoes catalytic cracking, cracked product is obtained under the action of catalyst, the cracked product and catalyst are separated by cyclone, and then the separated cracked product is further separated by fractionation system.

7. The method according to claim 1, wherein in the catalyst, a content of zeolite is 25%-40%.

8. The method according to claim 1, wherein the Y-type zeolite is a USY zeolite, or a USY zeolite modified with one or more elements of rare earth, phosphorus and alkaline earth metal.

9. The method according to claim 1, wherein the ZSM-5 zeolite is ZSM-5 zeolite modified with phosphorus and/or rare earth.

10. The method according to claim 1, wherein in the catalytic cracking reaction, the catalyst comprises 40%-60% of modified 10 MR zeolite, 20%-40% of clay, 10%-20% of alumina matrix and 1%-12% of binder on a dry basis.

11. The method according to claim 10, wherein the modified 10 MR zeolite is modified with IIIA group and phosphorus element through a post-modification method, with 10˜100:1 molar ratio of SiO₂/Al₂O₃=5% content of P₂O₅, and 0.1-3% oxide content of IIIA group element.

12. The method according to claim 10, wherein the 10 MR zeolite is one of the MFI zeolite, MEL zeolite, MFS zeolite, MWW zeolite and MTT zeolite.

13. The method according to claim 10, wherein the alumina matrix is built up of one or more of alumina, aluminum hydroxide monohydrate and aluminum hydroxide trihydrate.

14. The method according to claim 7, wherein the inorganic matrix is composed of alumina and/or modified alumina.

15. The method according to claim 7, wherein the binder is composed of one or more of alumina, silica, alumina-silica and phosphorus-alumina; the clay is selected from one or more of kaolinite, montmorillonite and attapulgite.

16. The method according to claim 1, wherein in the reaction, the mass ratio of catalyst to raw oil is 4-20.

17. The method according to claim 1, wherein the reaction outlet temperature is 490-650° C.; the weight hourly space velocity based on raw materials is 0.2-20 h⁻¹.

18. The method according to claim 1, wherein ethylene, propylene, gasoline and diesel oil are obtained, and the total yield of ethylene and propylene is more than 30%.
